(19) United States
(12) Patent Application Publication
(10) Pub. No.: US 2004/0215340 A1
(43) Pub. Date: Oct. 28, 2004

(54) Intraocular implant

(76) Inventors: Arthur Messner, Schnaittach (DE); Khalil Hanna, Paris (FR)

Correspondence Address: MCGLEW & TUTTLE, PC, 1 SCARBOROUGH STATION PLAZA, SCARBOROUGH, NY, 10510-0827, US

(21) Appl. No.: 10/486,528

(22) PCT Filed: Jul. 26, 2002
(86) PCT No.: PCT/EP02/08314

(30) Foreign Application Priority Data
Aug. 15, 2001 (DE) 101 39 027.0

(51) Int. Cl. A61F 2/16

(52) U.S. Cl. 623/6.37; 623/6.47; 623/6.49

(57) ABSTRACT

An accommodable implant for reception in the capsular bag of an eye, comprises an optical lens (2) with a lens plane (4) and a lens axis (5) extending perpendicular thereto and through the center of the lens (2); at least two haptics (3), with each haptic (3) extending radially outward from the lens (2), and being formed integrally with the lens (2), and comprising an arm (8) which is articulated to the lens (2) by a first joint (9), and comprising a supporting element (15) for support in the equatorial area of the capsular bag, which supporting element (15) is connected to the outer end of the arm (8).

The present invention relates to an accommodable implant for reception in the capsular bag of an eye according to the preamble of claim 1.

From literature, numerous accommodable lenses are known which usually have not been implemented in practice until now due to existing shortcomings. A detailed analysis of the state of the art can be found in PCT/FR01/00407.

An accommodable implant of the type mentioned above is known from EP 1 108 402 A2. Further accommodable implants are known from DE 199 38 590 A1 and WO 96/25126 A1.

The object of the present invention is to provide an accommodable implant for reception in the capsular bag of an eye which is easy to manufacture and fulfils its intended function.

This object is achieved by the features specified in claim 1.

Further advantageous embodiments of the present invention will become clear from the dependent claims.

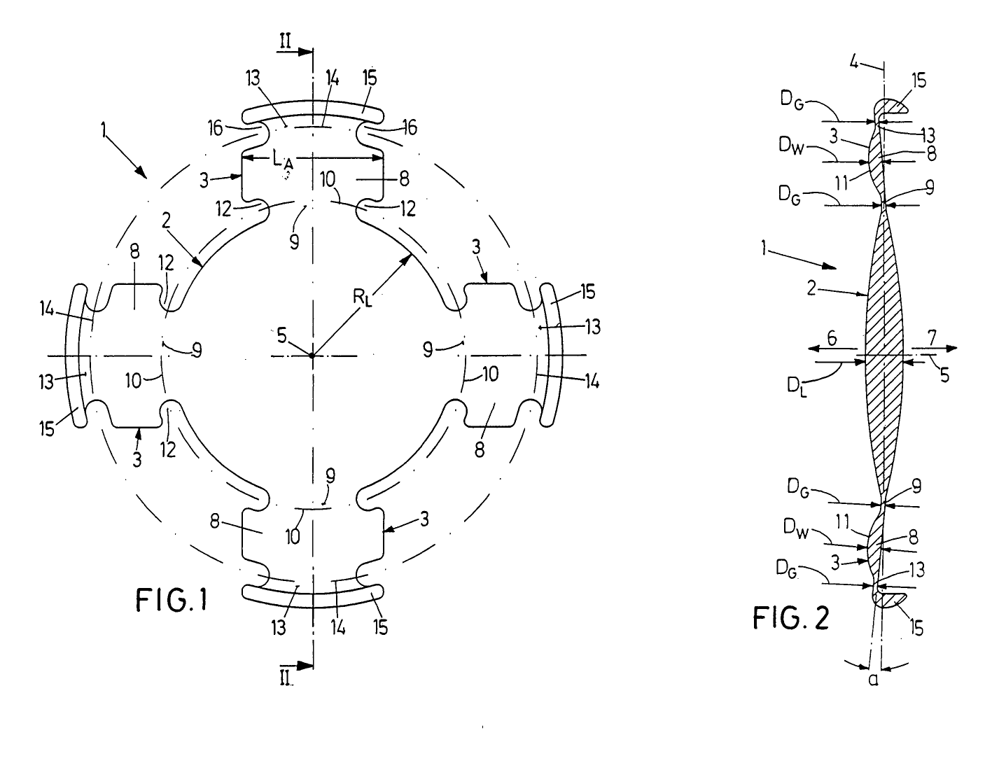

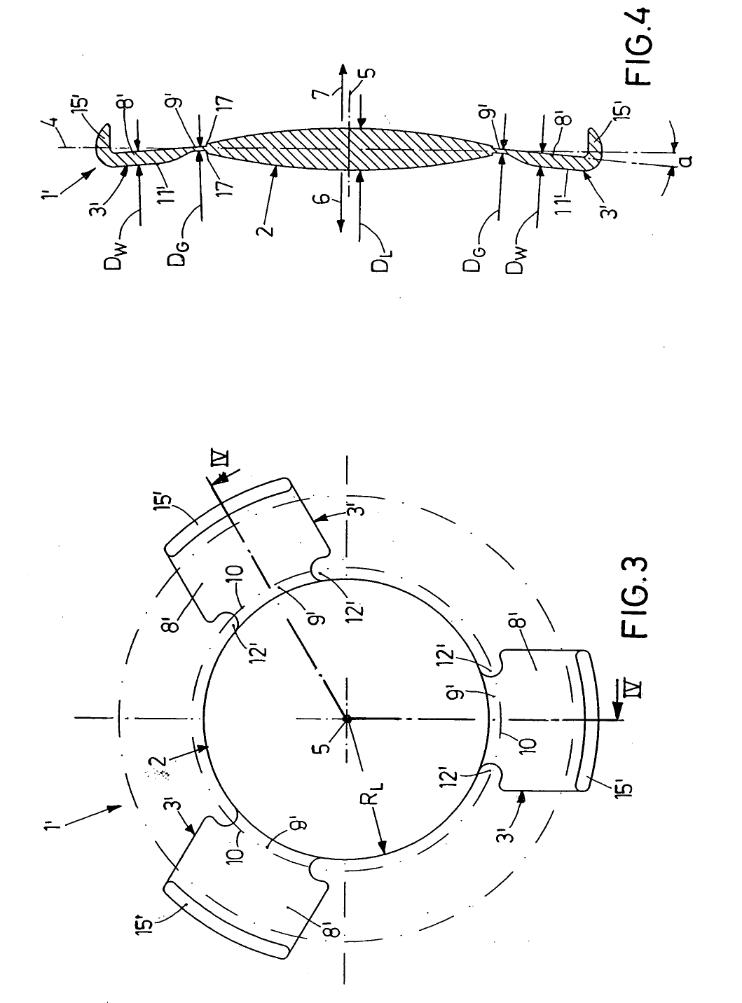

Further features and details of the present invention will become clear from the description of two example embodiments with reference to the drawing. The explanations with respect to FIGS. 3 to 5 are of importance only because they serve, together with FIGS. 1 to 2 and 6 to 7, to explain the first and the second example embodiment. In the drawing:

FIG. 1 is a plan view of an implant according to a first example embodiment;

FIG. 2 is a cross-sectional view along the sectional line II-II in FIG. 1;

FIG. 3 is a plan view of an implant;

FIG. 4 is a cross-sectional view along the sectional line IV-IV in FIG. 3;

FIG. 5 is a partial enlarged view of the cross-section according to FIG. 4;

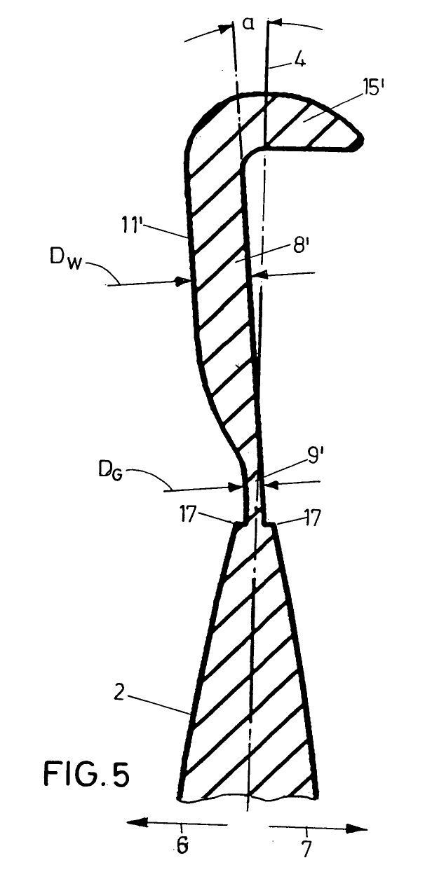

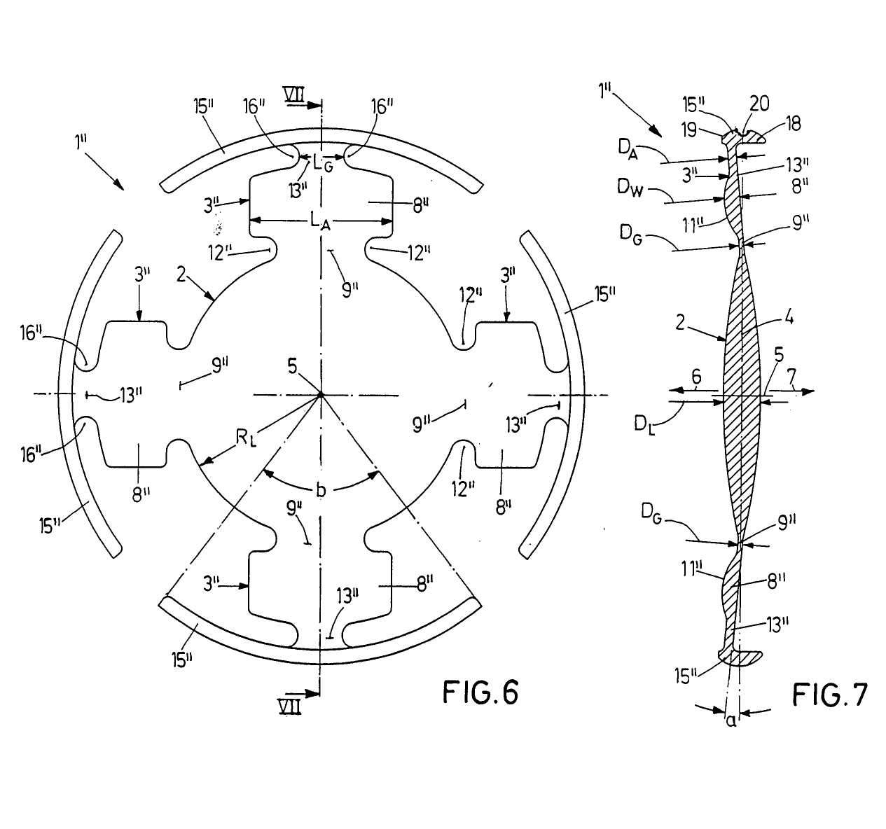

FIG. 6 is a plan view of an implant according to a second example embodiment; and

FIG. 7 is a cross-sectional view along the sectional line VII-VII in FIG. 6.

Now, a first embodiment of the present invention will be described with reference to FIGS. 1 and 2. An accommodable implant 1 for reception in the capsular bag of an eye comprises a central optical lens 2 and four arm-shaped haptics 3 which extend radially outward and are formed integrally with the lens. The haptics 3 are each offset from one another by 90°. It is also possible to provide another number of haptics. Advantageously, the haptics are evenly distributed along the periphery of the lens 2. The lens 2, as well as the haptics 3, is manufactured of a known material such as flexible acryl or a silicon elastomer. These materials are elastically deformable so that on the one hand they can be deformed in order that the implant 1 may be inserted in the capsular bag through a tiny incision in the patient's eye. On the other hand, the implants 1 have a shape memory causing them to reassume their original shape once inside the capsular bag. The lens 2 has a bi-convex shape although other lens shapes may be used as well. The lens 2 has a lens plane 4 extending through its equator and a lens axis 5 extending perpendicular to the lens plane and through the centre of the lens 2. In accordance with the implantation of the implant 1 in the capsular bag of the eye, the lens 2 has a posterior direction 6 extending parallel to the lens axis 5 towards the rear part of the eye and an anterior direction 7 extending opposite to the posterior direction towards the front part of the eye. The haptics 3 are inclined in the posterior direction 6 and form together with the lens plane 4 an angle a to which the following applies: 2°≦a≦25° and preferably a≈5°. In its central area, the lens has a thickness D_{L} to which the following applies: 0.4 mm≦D_{L}≦1.2 mm. The following applies to the radius R_{L} of the lens 2, i.e. half its diameter: 2.25 mm≦R_{L}≦3.25 mm.

The haptics 3 comprise an arm 8 as their central portion, which arm is pivotally linked to the lens 2 via a joint 9 formed as a film hinge. The joint 9 has a bending axis 10 arranged in the plane 4. In the area of the bending axis 10, the joints 9 have a thickness D_{G} to which the following applies: D_{G}≈70 μm to 90 μm. The arm 8 comprises a bulge 11 projecting in the direction 6, wherein in the area of the bulge 11 the arm 8 has a thickness D_{W} to which the following applies: D_{W}≈300 μm. Thus, the arm 8 is considerably more rigid than the joint 9. In the plane 4, the arm 8 has a width L_{A} roughly corresponding with the radius R_{L} of the lens 2. In the area of the joint 9, recesses 12 receding behind the arm 8 are provided to facilitate the pivotability of the arm 8 at the joint 9. The recesses 12 have a depth roughly corresponding with 20% of the width L_{A} of the arm 8. At the outer end of the arm 8, a further joint 13 formed as a film hinge and having a bending axis 14 is provided, which joint has a structure similar to that of the joint 9. A supporting web 15 formed as a supporting element is pivotally linked to the arm 8 via the joint 13. The supporting web 15 projects from the joint 13 in the direction 7. The supporting web 15 is curved in the plane 4 so that it can abut to the equatorial area of the capsular bag of an eye. In the area of the joint 13, recesses 16 are provided which recede behind the arm 8 and the supporting web 15 to facilitate the pivotability of the joint 13. In the cross-sectional view shown in FIG. 2, the supporting web 15 tapers in the direction 7 and has a curved outer periphery.

The joints 9 have a flexural strength B_{I} and the joints 13 have a flexural strength B_{A}. The flexural strength is defined in the usual way. The flexural strength is the product of the modulus of elasticity E and the geometrical moment of inertia I of the cross-section around the respective bending axis. Since in the case of the polymer materials used to manufacture the implant 1 the linear part of the stress-strain curve is very small, the modulus of elasticity E is determined by applying the provision set forth in the DIN 13316 standard, according to which the modulus of elasticity E represents the ratio between the stress increase in the range of 0.05% and 0.25% strain and this 0.2% strain increase given uninhibited cross-sectional deformation. Comprehensive measurements and computer simulations have shown, that an accommodable implant 1 fulfilling its intended function can only be obtained if defined limits are observed with respect to flexural strength. The sum S_{Z} of the flexural strengths B_{I} and B_{A} of all four haptics 3 must be ≦30.0 Nmm², in particular ≦20.0 Nmm², and particularly advantageously ≦11.2 Nmm². The following applies to the flexural strength B_{I} of each joint 9: B_{I}≦3.0 Nmm², particularly advantageously B_{I}≦1.6 Nmm². The following applies to the flexural strength B_{A} of a joint 13: B_{A}≦2.0 Nmm², particularly advantageously B_{A}≦1.2 Nmm². Only these flexural strengths ensure that in the event of a natural concentric contraction of the capsular bag a sufficient accommodation of the lens 2 will occur. In respect of the physiological background of the implantation of the implant 1 and the general functioning of an accommodable lens, reference is made to PCT/FR01/00407.

Now, the functioning of the implant 1 will be described. In the event that the natural lens of a human eye becomes turbid, the lens is removed from the capsular bag through a small lateral incision, and the implant 1, which has been folded for this purpose, is inserted in the capsular bag so that the supporting webs 15 support the implant in the equatorial plane of the capsular bag. The lens 2 is implanted such that its anterior direction 7 extends towards the front part of the eye. The arms 8 ensure that the front and rear capsular membranes remain separated from each other. If the front and rear capsular membranes would stick together, the elasticity of the lens capsule would be reduced to such an extent that only a minor accommodation could be achieved. When the eye's accommodation apparatus exerts forces from the outside on the supporting webs 15 in the direction towards the axis 5, the supporting web 15 pivots relative to the arm 8 while the arm 8 pivots relative to the lens 2 thus displacing the lens 2 in the anterior direction 7. Due to the parallel displacement of the lens plane 4, the image is sharply focused on the retina. The flexural strengths of the joints 9 and 13 are adjusted such that the eye's accommodation apparatus can accomplish an appropriate displacement of the lens 2 in the anterior direction 7. When the force exerted on the supporting webs 15 decreases, the lens 2 automatically returns to its initial state due to the shape memory of the joints 9 and 13. In the pivoted state of the haptics 3, the bulges 11 serve as stops for the posterior capsular membrane of the capsular bag.

Now, FIGS. 3 to 5 will be described. Identical elements are denoted by the same reference numerals used with the first embodiment to the description of which reference is made here. Structurally different but functionally similar elements are denoted by the same reference numerals followed by an inverted comma. The essential difference from the first embodiment is that the outer joint 13 is not provided and that the supporting web 15′ is directly connected to the arm 8′. Starting from the joint 9′ having the thickness D_{G}, the thickness of the arm 8′ increases up to the thickness D_{W} while then remaining constant up to the supporting web 15′. At the end of the joint 9′ facing the axis 5, on the upper and lower sides, an annular edge 17 having a rectangular cross-section is provided which forms the transition to the lens 2. The edge 17 acts as a barrier against the proliferation of lens epithelial cells which remain inside the lens capsule and cause secondary cataract. The development of fibrotic secondary cataract would considerably reduce the elasticity of the lens capsule which is necessary for accommodation so that the accommodation function could not be guaranteed any more. Therefore, the annular edges 17 are of eminent importance to avoid secondary cataract. The implant 1′ further differs from the implant 1 of the first embodiment in that only three haptics 3′ are provided which are offset from one another by a 120° angle. The joints 9′ have a flexural strength B_{E} per joint. The following applies to the sum S_{E} of the flexural strengths B_{E} of all joints 9′: S_{E}≦30.0 Nmm², in particular S_{E}≦20.0 Nmm², particularly advantageously S_{E}≦12.0 Nmm². The following applies to the individual flexural strengths B_{E}: B_{E}≦6.0 Nmm², in particular B_{E}≦4.0 Nmm². In the plan view shown in FIG. 3, the supporting web 15′ is flush with the lateral edges of the arms 8′.

The second embodiment functions essentially in the same way as the first embodiment. Unlike the first embodiment, a force exerted from outside on the supporting webs 15′ causes only the arm 8′ to pivot relative to the lens 2 thus displacing the lens plane 4 in the anterior direction 7 and enabling an accommodation. Now, a second embodiment of the present invention will be described with reference to FIGS. 6 and 7. Identical elements are denoted by the same reference numerals used with the first embodiment to the description of which reference is made here. Structurally different but functionally similar elements are denoted by the same reference numerals followed by two inverted commas. As in the first example embodiment, four haptics 3″ are provided which are offset from one another by 90°. The joints 9″ have essentially the same thickness D_{G} as in the first embodiment. The joints 13″ have a thickness DA larger than the thickness D_{G}. The bulge 11″ has essentially the same thickness D_{W} as in the first example embodiment. The proportions of the recesses 12″ which can be seen in the plan view (FIG. 6) essentially correspond with those of the recesses 12 of the first example embodiment. The recesses 16″ in the area of the joint 13″ recede much more than the corresponding recesses 16 in the first example embodiment. They are about twice as deep. Together with the thickness D_{A} of the joint 13″ which is larger than the thickness D_{G} of the joint 13, the same flexural strength B_{A} as in the first embodiment can be achieved. As a whole, the same provisions as in the first embodiment apply to the flexural strengths B_{I} and B_{A} as well as to the sum S_{Z} of the flexural strengths. An essential difference from the first embodiment relates to the shape of the supporting webs 15″. The supporting webs 15″ are formed as arcs resembling an annular sector having a centre angle b to which the following applies: b≈77°. This means that about 85% of the outer periphery of the implant 1″ is surrounded by the curved supporting webs 15″. Further, the supporting webs 15″ comprise a projection 18 projecting in the direction 7 and a projection 19 projecting in the direction 6, the projection 18 being about five times higher than the projection 19, relative to the axis 5. The projections 18 and 19 separate the front and rear capsular membranes of the lens capsule so that there is no contact between the capsular membranes. Further, the pro jection 18 enlarges the contact area between the haptic 3″ and the inner side of the lens capsule so that the resulting, radially inward force is effective on the side of the haptic 3″ which is shown to the left of the plane 4 in FIG. 7 and thus also next to the bending line of the joint 9″. For adjusting a flexural strength B_{A} of the joint 13″ comparable to that of the first example embodiment, the width L_{G} of the joint 13″ is smaller than the corresponding width of the joint 13. On the other hand, the thickness D_{A} of the joint 13″ is larger than the thickness D_{G} of the joint 13. The annular shape of the supporting webs 15″ helps to avoid the proliferation of lens epithelial cells which are present most of all in the equatorial area of the lens capsule. The interruptions between the supporting webs 15″ of adjacent haptics 3″ reduce strength. Thus, this embodiment combines the effect of a capsule tension ring and an accommodable intraocular lens. The annular supporting web 15″ acts as a barrier against the lens epithelial cells present in the equatorial area of the lens capsule. In addition, it enables an omnidirectional and crumple-free spreading of the two capsular membranes. In order to enhance the effect of the annular supporting web 15″, a small tangential groove 20 may be formed in its peripheral surface. This groove 20 may be closed at the front ends of the annular supporting webs 15″ to enhance the secondary cataract inhibiting effect. The lens epithelial cells are enclosed in this groove and thus cannot proliferate any more.

1. An accommodable implant for reception in the capsular bag of an eye, comprising
a. an optical lens (2) with a lens plane (4) and a lens axis (5) extending perpendicular to the lens plane (4) and through the center of the lens (2) and
b. at least two haptics (3; 3′; 3″), wherein each haptic (3; 3′; 3″)
i. extends radially outward from the lens (2),
ii. is formed integrally with the lens (2),
iii. comprises an arm (8; 8′; 8″) which is articulated to the lens (2) by a first joint (9; 9′; 9″), and
iv. comprises a supporting element (15; 15′; 15″) for support in the equatorial area of the capsular bag, which supporting element (15; 15′; 15″) is connected to the outer end of the arm (8; 8″; 8″).

2. An implant according to claim 1, characterized in that the supporting element (15; 15″) is pivotally linked to the arm (8; 8″) by a second joint (13; 13″).

3. An implant according to claim 1, characterized in that the first joint (9; 9′; 9″) and the second joint (13; 13″) are formed as film hinges.

4. An implant according to claim 1, characterized in that the lens has an anterior direction (7) extending parallel to the lens axis (5) and a posterior direction (6) extending opposite thereto.

5. An implant according to claim 4, characterized in that the arm (8; 8′; 8″) comprises a bulge (11; 11′; 11″) projecting in the posterior direction (6).

6. An implant according to claim 4, characterized in that the supporting element (15; 15′; 15″) projects relative to the arm (8; 8′; 8″) in the anterior direction (7).

7. An implant according to claim 4, characterized in that the arm (8; 8′; 8″) is inclined relative to the lens plane (4) by an angle a in the posterior direction (6).

8. An implant according to claim 1, characterized in that in the event that the supporting elements (15′) are not articulated to the respective arms (8′) the first joint (9′) has a flexural strength B_{E}.

9. An implant according to claim 8, characterized in that the following applies to the sum S_{E} of the flexural strengths B_{E} of the at least two first joints (9′): S_{E}≦30.0 Nmm².

10. An implant according to claim 9, characterized in that the following applies to the sum S_{E} of the flexural strengths B_{E} of the at least two first joints (9′): S_{E}≦20.0 Nmm².

11. An implant according to claim 9, characterized in that the following applies to the sum S_{E} of the flexural strengths B_{E} of the at least two first joints (9′): S_{E}≦12.0 Nmm².

12. An implant according to claim 9, characterized in that in the case of three haptics (3′) the following applies to the flexural strength B_{E} of each first joint (9′): B_{E}≦6.0 Nmm².

13. An implant according to claim 12, characterized in that in the case of three haptics (3′) the following applies to the flexural strength B_{E} of each first joint (9′): B_{E}≦4.0 Nmm².

14. An implant according to claim 2, characterized in that the flexural strength of each first joint (9; 9″) is B_{I} and the flexural strength of each second joint (13; 13″) is B_{A}.

15. An implant according to claim 11, characterized in that the following applies to the sum S_{Z} of the flexural strengths B_{I} and B_{A} of all joints (9, 13; 9″, 13″): S_{Z}≦30.0 Nmm².

16. An implant according to claim 15, characterized in that the following applies to the sum S_{Z} of the flexural strengths B_{I} and B_{A} of all joints (9, 13; 9″, 13″): S_{Z}≦20.0 Nmm².

17. An implant according to claim 15, characterized in that the following applies to the sum S_{Z} of the flexural strengths B_{I} and B_{A} of all joints (9, 13; 9″, 13″): S_{Z}≦11.2 Nmm².

18. An implant according to claim 11, characterized in that in the case of four haptics (3; 3″) the following applies to the flexural strength B_{I} of each first joint (9; 9″): B_{I}≦3.0 Nmm².

19. An implant according to claim 18, characterized in that in the case of four haptics (3; 3″) the following applies to the flexural strength B_{I} of each first joint (9; 9″): B_{I}≦1.6 Nmm².

20. An implant according to claim 13, characterized in that the following applies to the flexural strength B_{A} of each second joint (13; 13″): B_{A}≦2.4 Nmm².

21. An implant according to claim 20, characterized in that the following applies to the flexural strength B_{A} of each second joint (13; 13″): B_{A}≦1.2 Nmm².
